(19) United States
(12) Patent Application Publication
(10) Pub. No.: US 2004/0230165 A1
(43) Pub. Date: Nov. 18, 2004

(54) Anti-microbial barrier

(76) Inventors: Frank R. Prosl, Duxbury, MA (US); Brian K. Estabrook, Foxboro, MA (US); Paul J. Smith, Smithfield, RI (US); Adrian C. Ravenscroft, Rochester, MA (US)

Correspondence Address: Mark J. Pandiscio, Pandiscio & Pandiscio, 470 Totten Pond Road, Waltham, MA, 02154, US

(21) Appl. No.: 10/685,341

(22) Filed: Oct. 14, 2003

Related U.S. Application Data
(63) Continuation of application No. 09/766,416, filed on Jan. 19, 2001, now abandoned.
(60) Provisional application No. 60/177,107, filed on Jan. 20, 2000.

(51) Int. Cl. A61M 5/32

(52) U.S. Cl. 604/265

(57) ABSTRACT

A member for mounting concentrically on a catheter and for disposition about a skin opening so as to be contiguous with the surfaces of the opening, the member being of sponge-like material, and an anti-microbial fluid disposed in the member and leachable onto the skin and into the opening.

### REFERENCE TO PENDING PRIOR PATENT APPLICATION

This patent application claims benefit of pending prior U.S. Provisional Patent Application Ser. No. 60/177,107, filed Jan. 20, 2000 by Frank R. Prosl et al. for ANTI-MICROBIAL BARRIER, which patent application is hereby incorporated herein by reference.

### FIELD OF THE INVENTION

This invention relates to medical devices in general, and more particularly to anti-microbial barriers for preventing infection in skin punctures and the like.

### BACKGROUND OF THE INVENTION

It is known to provide catheters which extend from outside a body, through the skin, and into selected interior areas of the body. Such catheters are used in many medical procedures, including dialysis and chemotherapy.

A recurrent problem arises from the fact that as the catheter slides through an opening in the skin and enters the body, the outside wall of the catheter tends to carry with it the abutting peripheral portion of the opening, causing the opening to flex inwardly of the body, producing a generally annularly-shaped depression in the skin adjacent to the catheter. This depression becomes a relatively hidden and protected area for bacteria to gather and propagate, and from which the bacteria find access to the body interior.

Accordingly, there is a need for an anti-microbial medium adapted to prevent congregation of bacteria around the catheter and to discourage the transit of bacteria from the skin surface into the body hosting the catheter.

### SUMMARY OF THE INVENTION

Accordingly, one object of the present invention is to provide an anti-microbial barrier for preventing bacteria from accumulating around a catheter and from entering the body along the catheter.

Another object of the present invention is to provide a catheter assembly including an anti-microbial barrier.

Still another object of the present invention is to provide an anti-microbial barrier for preventing the formation of bacteria on, or adjacent to, a wound located on or in a body surface.

These and other objects are addressed by the present invention which, in one preferred form, comprises a tubular member for mounting concentrically on a catheter and for disposition in a skin opening so as to be substantially contiguous with the surfaces of the opening, the tubular member being of sponge-like material, and an anti-microbial fluid disposed in the member and leachable onto the skin and into the opening.

In accordance with a further aspect of the present invention, there is provided a catheter assembly comprising a catheter for insertion into a body through an opening in the body, a sponge-like tubular member mounted concentrically on the catheter for disposition in the skin opening so as to be substantially contiguous with the opening in the body, and an anti-microbial fluid disposed in the tubular member and leachable onto the skin and into the opening.

In accordance with a still further aspect of the present invention, there is provided a bandage-type barrier comprising a layer of sponge-like material, a first covering layer over a first surface of the sponge-like layer and defining an aperture for receiving an anti-microbial fluid, and adhesive disposed on the barrier for securing the barrier to a skin surface of a body with the second surface of the sponge-like layer disposed adjacent a wound in the skin surface.

These and other features of the present invention, including various novel details of construction and combinations of parts, will now be more particularly described with reference to the accompanying drawings. It will be understood that the particular embodiments hereinafter disclosed are provided by way of illustration only and not as limitations of the invention. The principles and features of this invention may be employed in various and numerous other embodiments without departing from the scope of the present invention.

### BRIEF DESCRIPTION OF THE DRAWINGS

Reference is made to the accompanying drawings in which are shown illustrative embodiments of the invention, from which its novel features and advantages will be apparent.

In the drawings:

FIG. 1 is an elevational, partly sectional, view of a prior art catheter assembly extending through a skin portion of a body;

FIG. 2 is similar to FIG. 1 and depicting one form of tubular member and catheter assembly illustrative of embodiments of the present invention;

FIG. 2A is a sectional view taken through line 2A-2A of FIG. 2;

FIG. 3 is similar to FIG. 2 but illustrative of an alternative embodiment of the present invention;

FIG. 4 is a sectional view of still another embodiment of the present invention;

FIG. 5 is a sectional view of yet another embodiment of the present invention;

FIG. 6 is a top plan view of one form of bandage illustrative of an alternative embodiment of the present invention; and

FIG. 7 is a sectional view, taken along line 7-7 of FIG. 6.

### DETAILED DESCRIPTION OF THE PREFERRED EMBODIMENTS

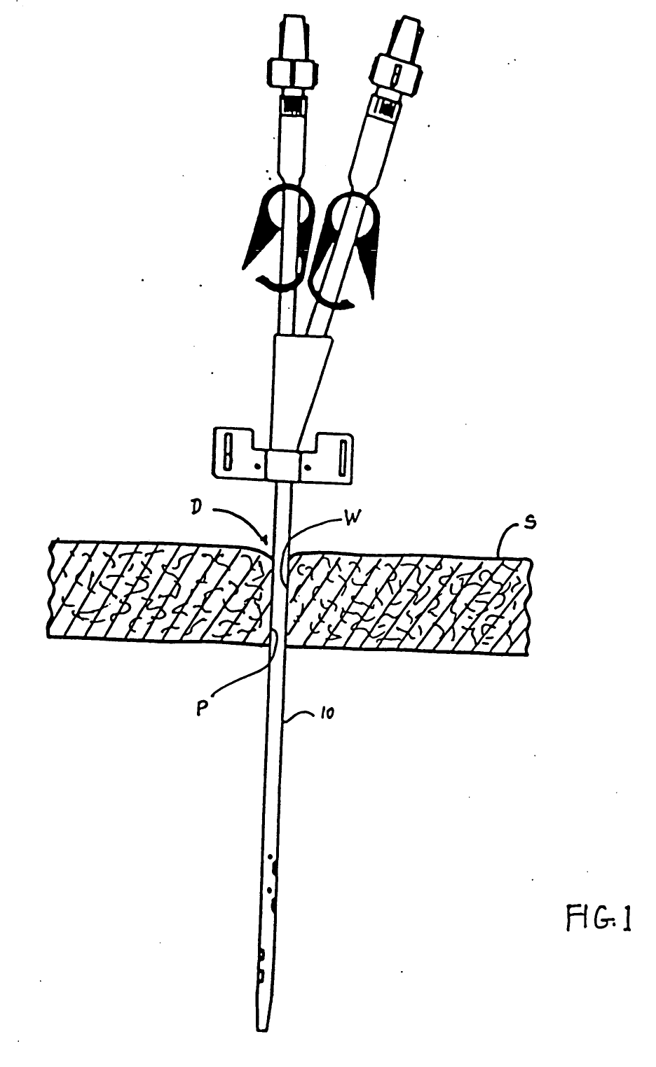

Referring to FIG. 1, it will be seen that a catheter 10 pushed through a body boundary layer of skin S typically causes formation of an annular depression D adjoining an internal wall W of an opening P in the skin S. Bacteria tend to congregate and propagate in the depression D and to migrate through the opening P into the body.

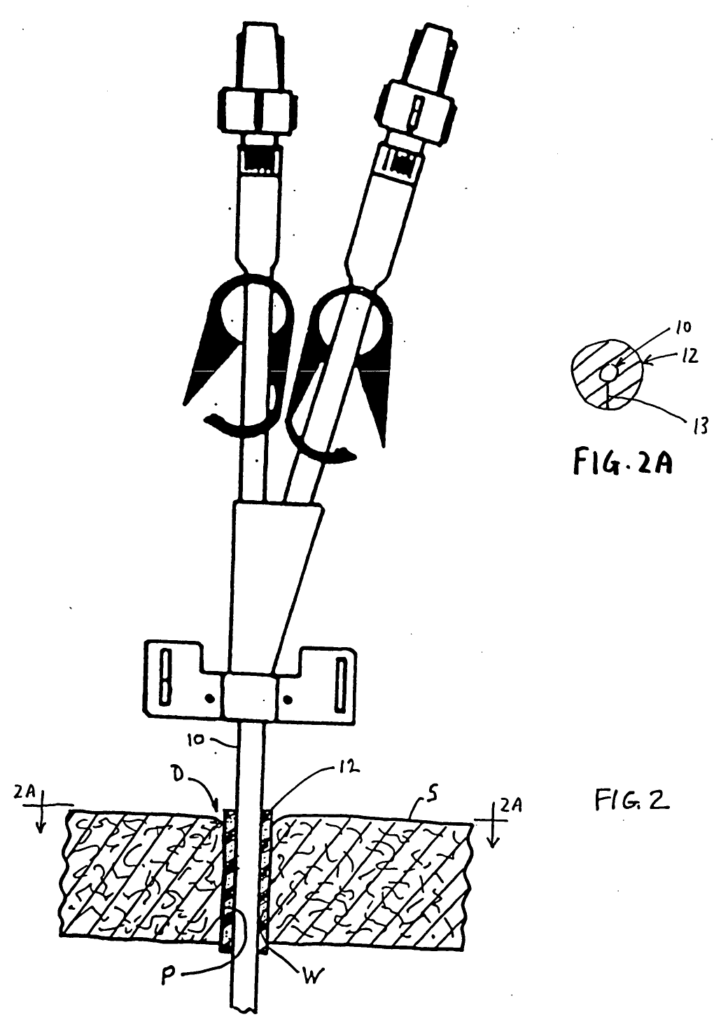

In accordance with the present invention, and looking now at FIG. 2, there is provided a tubular member 12 which is mounted on the catheter 10 for disposition, in use, in the skin opening P contiguous with the inside wall W of the opening P, that is, between the catheter 10 and the wall W of the opening P. The tubular member 12 is of a sponge-like material and is adapted to “soak up” fluids, retain the fluids, and permit the fluids to leach out therefrom over time and into the aforesaid depression D and opening P. As used herein, the term “sponge-like material” is meant to include any absorbable material capable of wicking fluid therefrom.

Tubular member 12 may be mounted on catheter 10 by a compression fit, and/or it may be secured to catheter 10 with an appropriate adhesive. Tubular member 12 may be manufactured in place or it may be mounted on catheter 10 later, e.g., at the time of catheter implantation, or at the time of catheter servicing, or when the tubular member 12 needs to be replaced. If desired, tubular member 12 may include a radial slit 13 to facilitate mounting to the catheter. See FIG. 2A. Tubular member 12 may extend along the length of catheter 10 so as to cover only the region passing through the skin or it may extend a longer distance, e.g., all the way down to an internal structure such as a blood vessel, an internal body cavity, etc.

The tubular member 12 contains an anti-microbial fluid. In one form of the invention, the anti-microbial fluid comprises Taurolidine or a Taurolidine-containing solution. And in one preferred form of the invention, the anti-microbial fluid comprises Taurolin® or a Taurolin-containing solution. Taurolin is a registered trademark of Geistlich Pharma AG of CH-6110 Wolhusen, Switzerland, and is a formulation comprising taurolidine (4,4′-methylene bis (tetrahydro-1,2,4-thiadiazine 1,1 dioxide). Taurolin exhibits a unique spectrum of antimicrobial activity, low toxicity, and high safety profile. If desired, the anti-microbial fluid may comprise an anti-microbial fluid such as that disclosed in U.S. Pat. No. 6,166,007 issued Dec. 26, 2000 to Klaus Sodemann for ANTIMICROBIAL LOCKS COMPRISING TAURINAMIDE DERIVATIVES AND CARBOXYLIC ACIDS AND/OR SALTS THEREOF, or an anti-microbial fluid such as that disclosed in U.S. patent application Ser. No. 09/483,966 filed Mar. 20, 2000 filed by Frank R. Prosl et al. for BIOCIDAL LOCKS, which patent and patent application are hereby incorporated herein by reference. In one preferred form of the invention, the anti-microbial fluid may comprise the Biolink CLS solution produced by Biolink Corporation of Norwell, Mass.

The sponge-like tubular member 12 readily absorbs the anti-microbial fluid and substantially retains the anti-microbial fluid, but permits a gradual leaching out of minute but effective quantities of the fluid over time. Eventually, the tubular member dries out, but a fresh supply of anti-microbial fluid can easily be injected, as by syringe, into the tubular member 12 which, again, absorbs the fluid. To this end, in one preferred form of the invention, tubular member 12 is preferably sized and positioned so that one end thereof extends flush with, or sits above, the outside surface of skin S, whereby the tubular member 12 may be easily accessed with a fresh supply of anti-microbial fluid. Alternatively, the dried out tubular member 12 may be replaced with a fresh tubular member 12 containing a fresh supply of anti-microbial fluid. The presence of the anti-microbial fluid essentially continuously cleanses the depression D and the opening P of bacteria.

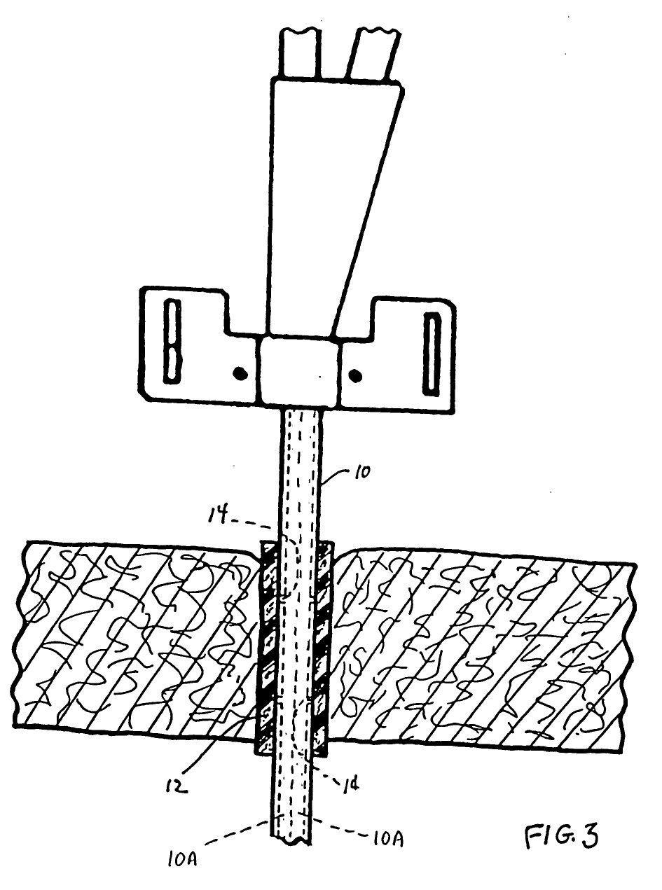

In some circumstances an anti-microbial fluid may be conveyed into the affected body by the working lumen(s) 10A of catheter 10, e.g., to provide a “catheter lock”, a procedure known in the dialysis art. In this situation, and referring now to FIG. 3, it will be seen that the catheter 10 may be provided with openings 14 through which the anti-microbial fluid may pass from working lumens 10A to the tubular member 12.

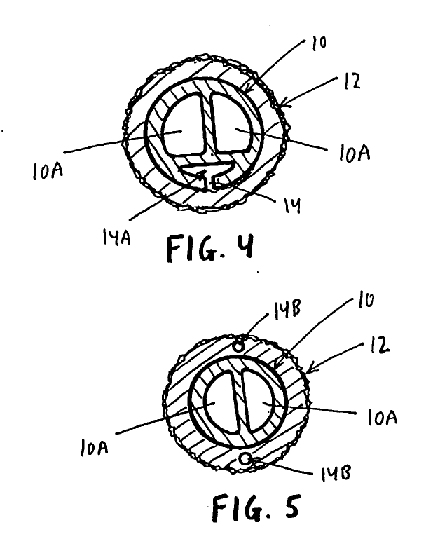

In another preferred form of the invention, and looking now at FIG. 4, the catheter 10 includes an additional replenishment lumen 14A which communicates with tubular member 12 via one or more openings 14. And in another form of the invention, and looking now at FIG. 5, replenishment lumens 14B may be provided directly in tubular member 12 itself.

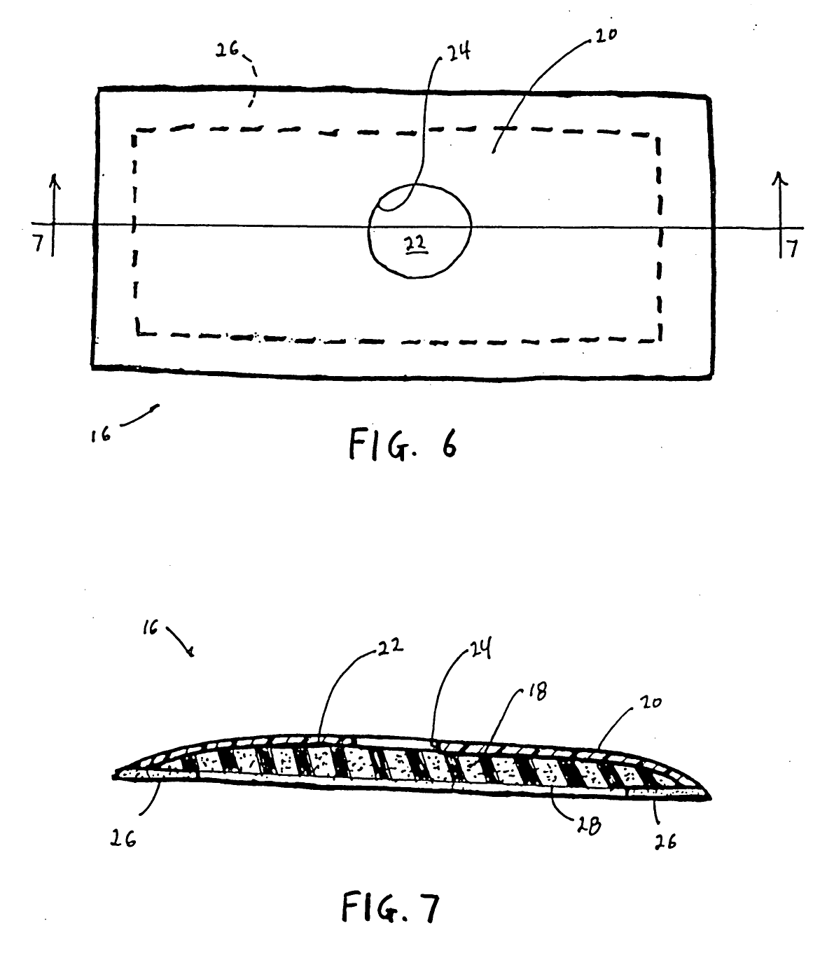

Referring next to FIGS. 6 and 7, it will be seen that the invention may assume the form of a bandage-type of barrier 16 including a layer 18 of sponge-like material. The bandage-type barrier 16 is provided with a first covering layer 20, preferably of a plastic or fabric material, extending over a first surface 22 of the sponge-like layer 18. The first covering layer 20 defines a central aperture 24 which exposes a portion of first surface 22 of sponge-like layer 18. Anti-microbial fluid may be injected into the sponge-like layer 18 by way of the aperture 24.

A second covering layer 26 extends over a portion of a second surface 28 of the sponge-like layer 18. An appropriate adhesive is disposed on or in the second covering layer 26 for securing the barrier 16 to a skin surface of a body. In operation, second surface 28 of sponge-like layer 18 is disposed adjacent to a puncture, or other wound, in a skin surface. The sponge-like layer 18 is injected with a suitable anti-microbial fluid, which is transmitted by the sponge-like layer 18 to the second surface 28 thereof, which is adjacent to the wound. Alternatively, if desired, second covering layer 26 could be omitted and the adhesive applied directly to the underside of sponge-like layer 18, or first covering layer 20 could be extended beyond the periphery of sponge-like layer 18 and the adhesive applied to this extended portion.

As used herein, the term “catheter” is intended to encompass any tubular structure extending into the body including, but not limited to, traditional catheters, shunts, cannulas, needles, etc.

If desired, the anti-microbial fluid may also include an antibiotic agent; or the anti-microbial fluid may be replaced by an antibiotic agent.

It will be understood that many additional changes in the details, materials, steps and arrangement of parts, which have been herein described and illustrated in order to explain the nature of the invention, may be made by those skilled in the art within the principles and scope of the invention.

What is claimed is:

1. A tubular member for mounting concentrically on a catheter and for disposition in a skin opening so as to be substantially contiguous with the surfaces of the opening, the tubular member being of sponge-like material, and an anti-microbial fluid disposed in the member and leachable onto the skin and into the opening.

2. A tubular member according to claim 1 wherein said tubular member includes a radial slit.

3. A catheter assembly comprising a catheter for insertion into a body through an opening in the body, a sponge-like tubular member mounted concentrically on the catheter for disposition in the skin opening so as to be substantially contiguous with the opening in the body, and an anti-microbial fluid disposed in the sponge-like tubular member and leachable onto the skin and into the opening.

4. A catheter assembly according to claim 3 wherein said tubular member includes a radial slit.

5. A catheter assembly according to claim 3 wherein said sponge-like tubular member is secured to said catheter.

6. A catheter assembly according to claim 3 wherein said sponge-like tubular member is replaceable.

7. A catheter assembly according to claim 3 wherein said sponge-like tubular member is replenishable by the catheter.

8. A catheter assembly according to claim 7 wherein said sponge-like tubular member is replenishable by a replenishment lumen contained within said catheter.

9. A catheter assembly according to claim 3 wherein said sponge-like tubular member includes a replenishment lumen.

10. A bandage-type barrier comprising a layer of sponge-like material, a first covering layer over a first surface of the sponge-like layer and defining an aperture for receiving an anti-microbial fluid, and adhesive disposed on the barrier for securing the barrier to a skin surface of a body with the second surface of the sponge-like layer disposed adjacent a wound in the skin surface.

11. A tubular member for mounting concentrically on a catheter and for disposition in a skin opening so as to be substantially contiguous with the surfaces of the opening, the tubular member being of sponge-like material, and an antibiotic agent disposed in the member and leachable onto the skin and into the opening.

12. A tubular member according to claim 11 wherein said tubular member includes a radial slit.

13. A catheter assembly comprising a catheter for insertion into a body through an opening in the body, a sponge-like tubular member mounted concentrically on the catheter for disposition in the skin opening so as to be substantially contiguous with the opening in the body, and an antibiotic agent disposed in the sponge-like tubular member and leachable onto the skin and into the opening.

14. A catheter assembly according to claim 13 wherein said tubular member includes a radial slit.

15. A catheter assembly according to claim 13 wherein said sponge-like tubular member is secured to said catheter.

16. A catheter assembly according to claim 13 wherein said sponge-like tubular member is replaceable.

17. A catheter assembly according to claim 13 wherein said sponge-like tubular member is replenishable by the catheter.

18. A catheter assembly according to claim 17 wherein said sponge-like tubular member is replenishable by a replenishment lumen contained within said catheter.

19. A catheter assembly according to claim 13 wherein said sponge-like tubular member includes a replenishment lumen.

20. A bandage-type barrier comprising a layer of sponge-like material, a first covering layer over a first surface of the sponge-like layer and defining an aperture for receiving an antibiotic agent, and adhesive disposed on the barrier for securing the barrier to a skin surface of a body with the second surface of the sponge-like layer disposed adjacent a wound in the skin surface.
